(19) United States
(12) Patent Application Publication
(10) Pub. No.: US 2026/0315225 A1
(43) Pub. Date: Oct. 15, 2026

(54) INTEGRATED TRANSCATHETER DEVICE FOR TAVR, TMVR, AND TTVR AORTIC VALVE REPLACEMENT WITH PMUT IMAGING

(71) Applicant: SoundCath, Inc., Canoga Park, CA (US)

(72) Inventors: Eric Stoppenhagen, Canoga Park, CA (US); Donald Masters, Sylmar, CA (US)

(73) Assignee: SoundCath, Inc., Canoga Park, CA (US)

(21) Appl. No.: 18/494,237

(22) Filed: Oct. 25, 2023

(51) Int. Cl. A61F 2/24; G06T 7/00

(52) U.S. Cl. CPC A61F 2/2436; A61F 2/243; G06T 7/00; G06T 2207/10132

(57) ABSTRACT

An integrated transcatheter valve replacement and Piezoelectric Micro-Machined Ultrasonic Transducer (pMUT) enabled ultrasonic imaging system, is disclosed. The system comprising a transcatheter valve replacement assembly having a longitudinal axis, a proximal end, and a distal end. The transcatheter valve replacement assembly comprises a self-expanding valve, a frame and leaflets. A micro-electromechanical (MEMS) based pMUT transducer having MEMS based pMUT array disposed within the distal end of the transcatheter valve replacement assembly. The MEMS based pMUT array comprises a substrate and a plurality of pMUT array elements arranged on the substrate. The integrated transcatheter valve replacement and pMUT enabled ultrasonic imaging system is used in left atrial appendage (LAA), atrial septal defect (ASD), transcatheter aortic valve replacement (TAVR), transcatheter mitral value replacement (TMVR), and transcatheter tricuspid valve replacement (TTVR) or other catheterized structural heart procedures.

### FIELD OF THE DISCLOSURE

The present disclosure relates generally to the field of cardiac valve replacement and ultrasonic imaging. The imaging includes left atrial appendage (LAA), atrial septal defect (ASD), transcatheter aortic valve replacement (TAVR), transcatheter mitral valve replacement (TMVR), and transcatheter tricuspid valve replacement (TTVR) or other catheterized structural heart procedures. More particularly, embodiments relate to catheters used in structural heart procedures (LAA, ASD, TAVR, TMVR, or TTVR) having an integrated distal microelectromechanical system (MEMS) based transducer for transmitting and receiving acoustic pulse information.

### BACKGROUND OF THE DISCLOSURE

With the increasing use of catheter-based procedures in cardiac interventions, the need for accurate imaging of the heart's anatomy has become critical. MEMS based Piezoelectric micro-machined ultrasonic transducer (pMUT) imaging has emerged as a promising technology for integrating a transcatheter aortic valve replacement (TAVR), a transcatheter mitral valve replacement (TMVR), a transcatheter tricuspid valve replacement (TTVR), a left atrial appendage (LAA), and an atrial septal defect (ASD) (together Structural Heart) with ultrasound imaging. MEMS based pMUT imaging uses a miniature ultrasound transducer that is small enough to fit into a catheter and is capable of providing high-resolution images of the heart's internal structures. The technology enables real-time imaging during Structural Heart procedures.

Aortic stenosis (AS) is the most common acquired heart valve disease in developed countries, affecting up to 10% of elderly patients. The prevalence of AS is expected to increase over the next decades with the increasing life expectancy in most developed countries. Indeed, the global number of people older than 80 is foreseen to triple and surpass 400 million by 2050, with AS prevalence expected to be growing at a similar rate. AS has a 50% mortality rate at 5 years from symptom onset if left untreated. Until recently, surgical aortic valve replacement (SAVR) represented the only definitive treatment for patients with AS, as medical therapy may only mitigate symptoms. Nonetheless, considering the frailty and the relevant burden of comorbidities of many elderly patients with symptomatic AS, a considerable portion of this population was left untreated, due to high or prohibitive surgical risk. The TAVR is originally conceptualized in the early 1990s, largely inspired by the pioneering experiences in the field of percutaneous transluminal coronary angioplasty.

Valve-in-valve (ViV) TMVR is another minimally invasive technique that has emerged as a viable treatment option for patients with degenerated mitral valve bioprosthesis who are at high risk for repeat surgical mitral valve replacement. The procedure involves inserting a catheter into the heart and deploying a new valve inside the old one, thus restoring normal blood flow. Several access strategies, including trans-apical, transseptal, trans-jugular, and trans-atrial access have been developed for ViV-TMVR. The preferred approach for the procedure has shifted over time from a trans-apical to a transseptal approach due to advancements in TMVR technology that have enabled smaller delivery catheters with greater flexibility.

Following different studies in animal models, the first TAVR procedure was performed in 2002 for the treatment of AS in a patient with several comorbidities and cardiogenic shock. Since then, TAVR has revolutionized the treatment of patients with severe, symptomatic AS, as randomized controlled trials have shown similar, if not superior, outcomes following TAVR as compared with the SAVR in selected patients. TAVR first emerged as a plausible treatment option for patients with AS at high or prohibitive surgical risk. Due to major advances in TAVR technologies, subsequent trials have shown that it is a safe and effective alternative to surgery for patients at intermediate-to-low surgical risk. The number of TAVR procedures is rapidly increasing, and the continuous expansion of the population deemed suitable for TAVR has corresponded with an impressive constant evolution in TAVR devices and materials. Indeed, these advances have significantly reduced periprocedural complications, making it safe to shorten hospital stay and improve long-term outcomes. Moreover, these progresses have resulted in a wide armamentarium at our disposal, including bioprosthesis presenting different dimensions, designs, and deliverability, providing the opportunity to select a device based on each patient’s clinical and anatomic characteristics. In addition, as TAVR comes of age, clinical indications for TAVR are gradually expanding, from elderly and comorbid patients affected by calcific AS to younger patients, with bicuspid aortic valve, bioprosthesis degeneration, and/or aortic regurgitation (AR), all conditions that potentially require devices with specific features.

The LAA described as the “most lethal human attachment,” is responsible for >90% of embolic strokes. In 2020, stroke was the fifth leading cause of death in the United States after heart disease, cancer, COVID-19, and unintentional injury. Atrial fibrillation (AF) is associated with a 4 to 5-fold increased risk of ischemic stroke and accounts for 25% of the 700,000 cerebrovascular accidents that occur in the United States annually. This translates to an average of $351.2 billion direct and indirect costs in the United States alone. It is projected that strokes related to AF will markedly increase in the future unless effective mitigation strategies are implemented. Historically, the mainstay of treatment for stroke prevention in AF has been oral anticoagulation (OAC). Large, randomized, controlled clinical studies of vitamin K antagonists (VKA), direct thrombin inhibitors, and factor-Xa inhibitors have established OACs as the standard of care for stroke prevention in AF. Although systemic anticoagulation has been known to be highly effective in mitigating stroke risk in patients with AF, there are patient, physician, and systemic barriers that make it difficult for many patients to sustain OAC therapy over time. These challenges led to a quest for alternative nonpharmacologic strategies particularly for high-risk patients who are intolerant to standard therapy.

The history of LAA occlusion (LAAO) or excision may be traced back to the mid-20th century when cardiac surgical techniques specifically involving the mitral valve were developed. Rheumatic mitral stenosis is notorious for causing embolic strokes especially with concomitant AF. Unsurprisingly, the earliest documented LAA excision procedures were in patients with rheumatic mitral stenosis undergoing cardiac surgery. In 1949, Madden described patients who underwent LAA excision as prophylaxis for recurring atrial thrombi. A meta-analysis of 23 studies noted that 57% of patients with valvular AF had thrombi localized in the LAA and extended into the left atrial cavity. In contrast, in patients with nonrheumatic AF, 91% of thrombi were localized in the LAA only (P90% of thrombi located in the LAA in nonvalvular AF, attention has focused on occlusion or exclusion of the LAA for the prevention of embolic stroke.

The occurrence of thrombi in the LAA during atrial fibrillation may be due to stagnancy of the blood pool in the LAA. The blood may still be pulled out of the left ventricle, however less effectively due to irregular contraction of the left atrium caused by atrial fibrillation. Therefore, instead of active support of the blood flow by a contracting left Altium and atrial appendage, filling of the left ventricle may depend primarily or solely on the suction effect created by the left ventricle. Further, the contraction of the left atrial appendage may be out of phase up to 180 degrees with the left ventricle, which creates significant resistance to desired flow of blood. Further still, most left atrial appendage geometries are complex and highly variable, with large irregular surface areas and ostium or opening compared to the depth of the left atrial appendage. These aspects as well as others may lead to high flow resistance of blood out the left atrial appendage.

In an effort to reduce the occurrence of thrombi formation within the left atrial and prevent thrombi from the blood stream from within the left atrial appendage, several medical devices has been developed that closes off the left appendage from the and /or circulatory system, thereby lowing the risk of stroke due to thrombolytic material entering the blood stream from the left atrial appendage.

The ASD is an opening or hole (defect) in the wall (septum) that separates the top two chambers of the heart (atria). This defect allows oxygen-rich blood to leak into the oxygen-poor blood chambers in the heart. ASD is a defect in the septum between the heart's two upper chambers. The septum is a wall that separates the heart's left ventricle and right ventricle. Since its first attempt in 1976 by Mills and King, transcatheter closure has become an accepted alternative to surgical repair for ostium secundum atrial septal defects (ASDs). The technique is commonly offered as first intention treatment. Safe and effective closure is achieved in at least 80% of the unselected ASD population. Septal Occluders are the standard of care for minimally invasive ASD closure. These double-disc occluders are comprised of Nitinol mesh and polyester material. The devices, inserted percutaneously, are deployed using a catheter and consist of a self-expanding double disk composed of the Nitinol mesh. The disks sit on both sides of the septal defect and occlude the defect. They are designed to securely oppose the septal wall on each side of the defect and create a platform for tissue in-growth after implantation.

### SUMMARY OF THE DISCLOSURE

By way of introduction, the preferred embodiments described below include an easy-to-use integrated left atrial appendage (LAA), or an atrial septal defect (ASD), or a transcatheter aortic valve replacement (TAVR), or a transcatheter mitral value replacement (TMVR), or a transcatheter tricuspid valve replacement (TTVR) and Piezoelectric Micro-Machined Ultrasonic Transducer (pMUT) (or other MEMS based transducer) ultrasonic imaging catheter system is disclosed. It should be noted the integrated a left atrial appendage (LAA), an atrial septal defect (ASD), a transcatheter aortic valve replacement (TAVR), a transcatheter mitral value replacement (TMVR), or a transcatheter tricuspid valve replacement (TTVR) may be transcatheter aortic valve replacement (TAVR) catheters, transcatheter mitral valve replacement TMVR catheters, left atrial appendage (LAA) catheter, atrial septal defect (ASD) catheter or transcatheter tricuspid valve replacement or repair (TTVR) catheters. The pMUT ultrasonic imaging catheter system comprises a pMUT transducer enabled imaging and integrated LAA, ASD, TAVR, TMVR, TTVR catheter having a longitudinal axis, a proximal end, and a distal end. Further, an ultrasonic pMUT transducer array is disposed within the distal end of the pMUT imaging and integrated LAA, ASD, TAVR, TMVR, TTVR catheter. The ultrasonic pMUT transducer array comprises a plurality of pMUT transducer array elements arranged on a substrate. It may be noted that the plurality of pMUT transducer array elements corresponds to a micro-electromechanical (MEMS) based pMUT. Further, the integrated LAA, ASD, TAVR, TMVR, TTVR and pMUT ultrasonic imaging catheter system comprises a catheter shaft connected at one end to a handle assembly and at other end to the ultrasonic pMUT transducer array and integrated LAA, ASD, TAVR, TMVR, TTVR assembly. The catheter shaft encloses an electronic flexible cable which is in communication with at least one signal trace and is configured to: direct each of the plurality of pMUT transducer array elements, via the at least one signal trace, to transmit and receive, with respect to heart, ultrasound beams having a bandwidth including a predetermined fundamental mode vibration of each of the plurality of pMUT transducer array elements, such that a single array element may transmit and receive multiple fundamental mode vibrations simultaneously; receive at least one signal from the plurality of pMUT transducer array elements based on transmitting and receiving at least one ultrasound beam of the ultrasound beams, and construct at least one image of at least a portion of the heart based on the at least one signal.

In one embodiment, the integrated TAVR, TMVR, TTVR assembly comprises a self-expanding valve with nitinol frame and Bovine or Porcine leaflets. In another embodiment, the integrated TAVR, TMVR, TTVR assembly comprises a balloon expandable valve with a stainless steel alloy and Bovine or Porcine leaflets.

In one embodiment, the pMUT ultrasonic imaging catheter system comprises integrated TAVR, TMVR, TTVR having an expanding valve, a frame and leaflets.

In some embodiments, the transcatheter valve replacement assembly comprises a catheter having 1 to a plurality of pMUT linear arrays. The catheter comprises a pMUT circular array.

According to another aspect of the invention, an integrated LAA, ASD, TAVR, TMVR, TTVR and pMUT ultrasonic imaging system, is disclosed. The system comprises an integrated LAA, ASD, TAVR, TMVR, TTVR assembly having a longitudinal axis, a proximal end, and a distal end. Further, the system comprises a micro-electromechanical (MEMS) based pMUT array disposed within the distal end of the integrated LAA, ASD, TAVR, TMVR, TTVR assembly. The MEMS based pMUT array comprises a substrate and a plurality of MEMS based pMUT array elements arranged on the substrate. Further, the system comprises an electronic flex cable connected at one end to a handle assembly and at other end to the MEMS based pMUT array. The electronic flex cable is in communication with at least one signal trace, and is configured to: direct each of the plurality of MEMS based pMUT array elements, via the at least one signal trace, to transmit and receive, with respect to heart, ultrasound beams; receive at least one signal from the plurality of MEMS based pMUT array elements based on transmitting and receiving at least one ultrasound beam of the ultrasound beams; and construct at least one image of at least a portion of the heart based on the at least one signal. The ultrasound beams having a bandwidth including a predetermined fundamental mode vibration of each of plurality of pMUT array elements, such that a single array element transmits and receives multiple fundamental mode vibrations simultaneously. In some embodiments, the pMUT imaging and transcatheter aortic valve replacement contains one to a plurality of pMUT arrays.

According to another aspect of the invention, a method of positioning a prosthetic implant within a heart is disclosed. The method comprises steps such as, at first, advancing together a delivery catheter and an introducer catheter preassembled over the delivery catheter into a patient's vascular system. The delivery catheter comprises a prosthetic valve and a distal tip inserted directly into the access vessel to dilate the distal tip to the access vessel for the introducer catheter. The prosthetic valve corresponds to the prosthetic implant or a cardiovascular prosthetic implant. It may be noted that during advancement, an outer diameter of the distal end of the delivery catheter is greater than an inner diameter of a distal end of the introducer catheter. The introducer catheter comprises a hemostasis valve assembly at a proximal end of the introducer catheter. The method comprises advancing a prosthetic valve to a position proximate a native valve of the heart, the prosthetic valve being at least partially disposed within a distal end of the delivery catheter during advancement of the introducer catheter and deploying the prosthetic valve.

In one embodiment, the delivery catheter is advanced over an aortic arch and past the aortic valve, the position of an outer tubular member of the delivery catheter relative to the introducer catheter is maintained by adjusting a seal assembly.

According to an embodiment, an integrated transcatheter left atrial appendage (LAA) closure and Piezoelectric Micro-Machined Ultrasonic Transducer (pMUT) ultrasonic imaging system is disclosed. The system comprises an integrated transcatheter LAA closure assembly having a longitudinal axis, a proximal end, and a distal end, and a micro-electromechanical (MEMS) based pMUT transducer having MEMS based pMUT array disposed within the distal end of the integrated transcatheter LAA closure assembly. The MEMS based pMUT array comprises a substrate and a plurality of pMUT array elements arranged on the substrate.

In some embodiments, a catheter shaft connected at one end to a handle assembly and at other end to the MEMS based pMUT array and the integrated transcatheter LAA closure assembly.

In some embodiments, the MEMS based pMUT array and the integrated transcatheter LAA closure assembly comprises a steering control unit positioned within the handle assembly, for articulating a distal tip of the integrated transcatheter LAA closure assembly and aligning face of the MEMS based pMUT array towards internal views including an anterior position or a posterior position and right or left position of the tissue.

In some embodiments, the distal tip of the MEMS based pMUT array is coated with a material to provide electrical isolation and transmission of ultrasound signals.

In some embodiments, the MEMS based pMUT array and the integrated transcatheter LAA closure assembly is coupled to a dongle, and the dongle is configured to communicate ultrasound transmit pulses and ultrasound receive waveforms between the pMUT array and the ultrasound imaging system.

In some embodiments, each of the plurality of pMUT array elements having transducer cells of multiple diameters, to achieve a wide bandwidth.

In some embodiments, each of the plurality of pMUT array elements is a linear phased array.

In some embodiments, each of the plurality of pMUT array elements is a pMUT circular array.

In some embodiments, the integrated transcatheter LAA closure assembly comprises a catheter having 1 to a plurality of pMUT arrays. In some embodiments, the catheter includes one pMUT circular array.

In some embodiments, the system is deployed into the heart chambers.

According to another embodiment, an integrated transcatheter atrial septal defect (ASD) closure and Piezoelectric Micro-Machined Ultrasonic Transducer (pMUT) ultrasonic imaging system is disclosed. The system comprises an integrated transcatheter ASD closure assembly having a longitudinal axis, a proximal end, and a distal end, and a micro-electromechanical (MEMS) based pMUT transducer having MEMS based pMUT array disposed within the distal end of the integrated transcatheter ASD closure assembly. The MEMS based pMUT array comprises a substrate and a plurality of pMUT array elements arranged on the substrate.

In some embodiments, a catheter shaft is connected at one end to a handle assembly and at other end to the MEMS based pMUT array and the integrated transcatheter ASD closure assembly.

In some embodiments, the MEMS based pMUT array and the integrated transcatheter ASD closure assembly comprises a steering control unit positioned within the handle assembly, for articulating a distal tip of the integrated transcatheter ASD closure assembly and aligning face of the MEMS based pMUT array towards internal views including an anterior position or a posterior position and right or left position of the tissue.

In some embodiments, the MEMS based pMUT array and the integrated transcatheter ASD closure assembly is coupled to a dongle, and the dongle is configured to communicate ultrasound transmit pulses and ultrasound receive waveforms between the pMUT array and the ultrasound imaging system.

In some embodiments, the integrated transcatheter ASD closure assembly comprises a catheter having 1 to a plurality of pMUT linear arrays. The catheter comprises a pMUT circular array.

According to an embodiment, an integrated transcatheter tricuspid valve replacement or repair (TTVR) and Piezoelectric Micro-Machined Ultrasonic Transducer (pMUT) ultrasonic imaging system is disclosed. The system comprises a transcatheter TTVR assembly having a longitudinal axis, a proximal end, and a distal end. The transcatheter TTVR assembly comprises a self-expanding valve, a frame and leaflets. Further, the system comprises a micro-electromechanical (MEMS) based pMUT transducer having MEMS based pMUT array disposed within the distal end of the transcatheter TTVR assembly. The MEMS based pMUT array comprises a substrate and a plurality of pMUT array elements arranged on the substrate.

### BRIEF DESCRIPTION OF THE DRAWINGS

The accompanying drawings illustrate various embodiments of systems, methods, and embodiments of various aspects of the disclosure. Any person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another and vice versa. Furthermore, elements may not be drawn to scale. Non-limiting and non-exhaustive descriptions of the present disclosure are described with reference to the following drawings. The components in the figures are not necessarily to scale, emphasis instead being placed upon the illustrated principles.

Various embodiments will hereinafter be described in accordance with the appended drawings, which are provided to illustrate and not to limit the scope of the disclosure in any manner, wherein similar designations denote similar elements.

FIG. 1 illustrates a prior art imaging system, for acquiring two-dimensional image information, according to an embodiment of the present disclosure.

FIG. 2 illustrates the prior art imaging system, for acquiring the two-dimensional image information, according to an embodiment of the present disclosure.

FIG. 3 illustrates a schematic diagram of a left atrial appendage (LAA), an atrial septal defect (ASD), a transcatheter aortic valve replacement (TAVR), a transcatheter mitral value replacement (TMVR), and a transcatheter tricuspid valve replacement (TTVR) and a Piezoelectric Micro-Machined Ultrasonic Transducer (pMUT) ultrasonic imaging catheter system, according to an embodiment of the present disclosure.

FIG. 4 illustrates a multi-channel electronic communication between a pMUT imaging and mapping device and a pMUT imaging and the LAA, the ASD, the TAVR, the TMVR, or the TTVR, according to an embodiment of the present disclosure.

FIG. 5 illustrates a sectional view of a distal end of the pMUT imaging assembly with a plurality of pMUT transducer array elements, according to an embodiment of the present disclosure.

FIG. 6 illustrates heart chambers and valves, according to an embodiment of the present disclosure.

FIG. 7A illustrates a step of partially deploying and positioning an artificial valve implant, according to an embodiment of the present disclosure.

FIG. 7B illustrates a second step of partially deploying and positioning the artificial valve implant, according to an embodiment of the present disclosure.

FIG. 7C illustrates a third step of partially deploying and positioning the artificial valve implant, according to an embodiment of the present disclosure.

FIG. 7D illustrates a fourth step of partially deploying and positioning the artificial valve implant, according to an embodiment of the present disclosure.

FIG. 8 illustrates a side perspective view of the introducer catheter and the integrated TAVR, TMVR, and TTVR assembly of FIGS. 7A-7D with the implant inside of an outer sheath jacket, according to an embodiment of the present disclosure.

FIG. 9 illustrates a perspective view of a distal portion of the pMUT imaging and integrated TAVR, TMVR, and TTVR assembly in a partially deployed state, according to an embodiment of the present disclosure.

FIG. 10 illustrates a perspective view of the pMUT imaging and integrated TAVR, TMVR, and TTVR assembly, with a plurality of linear pMUT imaging arrays arranged in a linear fashion, according to an embodiment of the present disclosure.

FIG. 11 illustrates a perspective view of the pMUT imaging and integrated TAVR, TMVR, and TTVR assembly, with a plurality of circular pMUT imaging arrays arranged in a circular fashion, according to an embodiment of the present disclosure.

FIG. 12 illustrates heart chambers and valves, with a Watchman or Amulet deployed into the LAA, according to an embodiment of the present disclosure.

FIG. 13A illustrates a catheter sheath with marker band(s) and a circular array at a distal end, according to an embodiment of the present disclosure.

FIG. 13B illustrates a catheter sheath with marker band(s) and a linear array at the distal end, according to an embodiment of the present disclosure.

FIG. 14 illustrates a catheter sheath deploying a watchman into the LAA, according to an embodiment of the present disclosure.

FIG. 15 illustrates heart chambers with an Arterial Septum closure device, according to an embodiment of the present disclosure.

FIG. 16A illustrates deployment of the Arterial Septum closure device, according to an embodiment of the present disclosure.

FIG. 16B illustrates another view for deployment of the Arterial Septum closure device, according to an embodiment of the present disclosure.

FIG. 17A illustrates a perspective view of continuing deployment of the Arterial Septum closure device, according to an embodiment of the present disclosure.

FIG. 17B illustrates another perspective view of continuing deployment of the Arterial Septum closure device, according to an embodiment of the present disclosure.

FIG. 17C illustrates a side perspective view of the deployed Arterial Septum closure device, according to an embodiment of the present disclosure.

FIG. 18A illustrates an addition of PMUT imaging to the catheter with a pMUT circular array, according to an embodiment of the present disclosure.

FIG. 18B illustrates the addition of PMUT imaging to the catheter with a pMUT linear array, according to an embodiment of the present disclosure.

### DETAILED DESCRIPTION

The components of the embodiments as generally described and illustrated in the figures herein may be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context dictates otherwise. Although any systems and methods similar or equivalent to those described herein may be used in the practice or testing of embodiments of the present disclosure, the preferred systems and methods are now described. The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

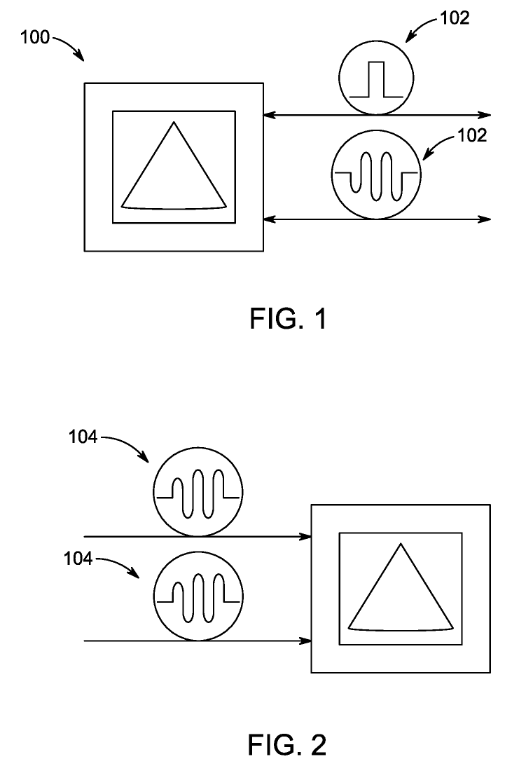

FIGS. 1 and 2 illustrate a prior art imaging system 100. The imaging system 10 provides an ultrasound transmit pulse 102 and an ultrasound receive path 104, for connection to an ultrasonic transducer (not shown). The ultrasound transmit pulse 102 may transmit ultrasound signals from the imaging system 100 towards an object such as heart of a patient. Further, the ultrasound receive path 104 may create a waveform based at least on the ultrasound signals. Thereafter, the imaging system 100 may convert the received ultrasound signals or ultrasound information to a two-dimensional (2D) image of the object or a portion of the object.

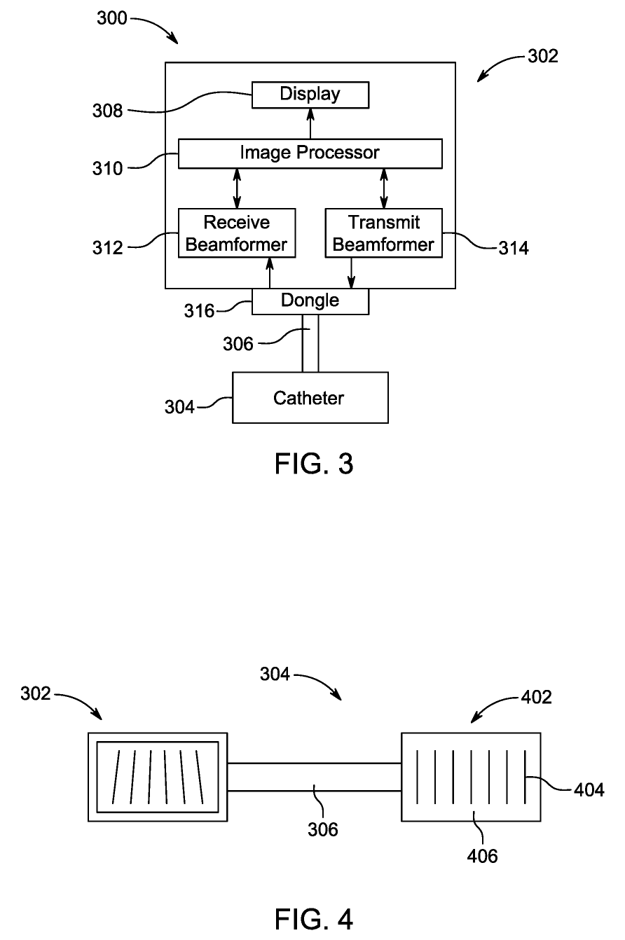

FIG. 3 illustrates a schematic diagram of an integrated left atrial appendage (LAA), an atrial septal defect (ASD), a transcatheter aortic valve replacement (TAVR), a transcatheter mitral value replacement (TMVR), a transcatheter tricuspid valve replacement (TTVR) and a Piezoelectric Micro-Machined Ultrasonic Transducer (pMUT) ultrasonic imaging catheter system 300, according to an embodiment of the present disclosure.

In one embodiment, the integrated LAA, ASD, TAVR, TMVR, and TTVR and pMUT ultrasonic imaging catheter system 300 may utilize a micro-electromechanical (MEMS) pMUT transducer array defined as pMUT or other types of MEMS transducers, interconnected using matched flexible circuits. It may be noted that the use of the high-density flexible circuits may enable highly repeatable and stable transmission and return signals. Further, the high-density flexible circuit transmission lines may transmit electrical energy from one end to another distal end of the integrated LAA, ASD, TAVR, TMVR, and TTVR assembly and pMUT ultrasonic imaging catheter system 300. Hereinafter, the integrated LAA, ASD, TAVR, TMVR, and TTVR assembly and pMUT ultrasonic imaging catheter system 300 and an integrated ultrasonic imaging system 300 may be used interchangeably.

The integrated ultrasonic imaging system 300 may comprise a pMUT imaging device 302 linked to a pMUT imaging and integrated LAA, ASD, TAVR, TMVR, and TTVR assembly 304 via a communication channel 306. In some embodiments, the pMUT imaging and integrated LAA, ASD, TAVR, TMVR, and TTVR assembly 304 may be referred as a pMUT imaging and integrated LAA, ASD, TAVR, TMVR, and TTVR catheter 304. The pMUT imaging device 302 may comprise a display 308, an image processor 310, a receive beamformer 312, a transmit beamformer 314 and a dongle 316. The pMUT imaging and integrated LAA, ASD, TAVR, TMVR, and TTVR assembly 304 may be disposed within a chamber of a heart of a patient and the pMUT imaging device 302 may receive at least one signal from the pMUT imaging and integrated LAA, ASD, TAVR, TMVR, and TTVR assembly 304. The at least one signal may be communicated from the pMUT imaging and integrated LAA, ASD, TAVR, TMVR, and TTVR assembly 304 to the pMUT imaging device 302 via an electronic flex cable (not shown) connected to the dongle 316.

The image processor 310 may be configured to generate a two-dimensional (2D) image according to data received from the pMUT imaging and integrated LAA, ASD, TAVR, TMVR, and TTVR assembly 304. In one embodiment, the image processor 310 may be configured to receive a focused signal from the receive beamformer 312. The image processor 310 may render the data to construct an image or sequence of images. In one embodiment, the image may be three-dimensional (3D) representation, such as a two-dimensional image rendered from a user or a processor selected viewing direction. In one embodiment, the image processor 310 may be a detector, filter, processor, application-specific integrated circuit, field-programmable gate array, digital signal processor, control processor, scan converter, three-dimensional image processor, graphics processing unit, analog circuit, digital circuit, or combinations thereof. The image processor 310 may receive beamformed data and may generate images, to display on the display 308. It may be noted that the generated images are associated with a two-dimensional (2D) scan. Alternatively, the generated images may be three-dimensional (3D) representations.

The image processor 310 may be programmed for hardware accelerated two-dimensional re-constructions. The image processor 310 may store processed data of the at least one signal and a sequence of images in a memory. In one embodiment, the memory may be a non-transitory computer-readable storage media. The instructions for implementing the processes, methods and/or techniques discussed herein are provided on the computer-readable storage media or memories, such as a cache, buffer, RAM, removable media, hard drive, or other computer-readable storage media. Non-transitory computer-readable storage media includes various types of volatile and non-volatile storage media. The functions, acts, or tasks illustrated in the figures or described herein are executed in response to one or more sets of instructions stored in or on a computer readable storage media. The functions, acts, or tasks are independent of the particular type of instruction sets, storage media, processor, or processing strategy and may be performed by software, hardware, integrated circuits, firmware, micro code, and the like, operating alone or in combination.

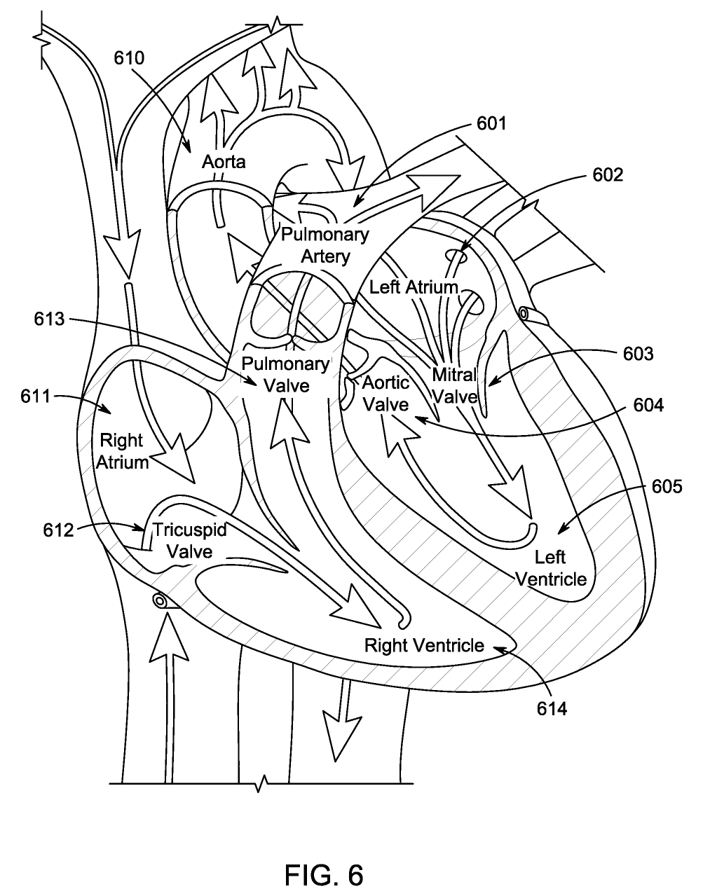

The pMUT imaging and integrated LAA, ASD, TAVR, TMVR, and TTVR assembly 304 may be in electronic communication with the pMUT imaging and mapping device 302 for transmission and receiving of ultrasound signals to and from an arterial wall of a vascular system. In one embodiment, the pMUT imaging and integrated LAA, ASD, TAVR, TMVR, and TTVR assembly 304 may be configured to visualize standard echocardiography views of the heart, such as in a standard version, a right atrium may be visualized. In some embodiments, the pMUT imaging and integrated ASD assembly 304 may be employed in transseptal catheterization for several percutaneous interventions, including left heart catheter mapping, atrial septal defect closure for effective alternative to surgical intervention. In one embodiment, the pMUT imaging and integrated LAA, ASD, TAVR, TMVR, and TTVR assembly 304 may comprise a body (not shown) having a longitudinal axis, a proximal end, a distal end, a handle assembly, a catheter shaft, an electronic flex cable, and a distal tip, as shown in FIG. 6.

Referring to FIG. 4, a multi-channel electronic communication between the pMUT imaging device 302 and the pMUT imaging and integrated LAA, ASD, TAVR, TMVR, and TTVR assembly 304 is disclosed, according to an embodiment of the present disclosure.

The pMUT imaging and transcatheter aortic valve replacement 304 may comprise a MEMS based pMUT array 402 coupled to the pMUT imaging and mapping device 302 via the catheter shaft (not shown) and the dongle 316. The dongle 316 may be referred as a communication channel 306 connected to the catheter shaft.

The MEMS based pMUT array 402 may comprise a plurality of pMUT array elements 404 arranged on a substrate 406. The catheter comprises a pMUT circular array. In one example, the pMUT imaging and transcatheter aortic valve replacement 304 may contain 1 pMUT array. In another example, the pMUT imaging and transcatheter aortic valve replacement 304 may contain a plurality of pMUT arrays. Further, each of the plurality of pMUT array elements 404 may provide a wide bandwidth of an individual focused beam. In some embodiments, the plurality of pMUT array elements 404 may include 32 64 or 96 array elements. The MEMS based pMUT array 402 may be coupled to the pMUT imaging device 302 using the dongle 316, as described earlier. The MEMS based pMUT array 402 disposed within the distal end of the pMUT imaging and integrated LAA, ASD, TAVR, TMVR, and TTVR assembly 304 may transmit the at least one signal via the electronic flex cable inside the catheter shaft to the pMUT imaging device 302. The at least one signal may be the acoustic echo transmitted from the MEMS based pMUT array 402. It may be noted that the acoustic echo of acoustic energy may be received from a face of the MEMS based pMUT array 402 and received at the image processor 310.

The ultrasound beams may have a bandwidth including a predetermined fundamental mode vibration of each of the plurality of pMUT array elements 404, such that a single array element may transmit and receive multiple fundamental mode vibrations simultaneously. It may be noted that the plurality of pMUT array elements 404 may transmit and receive the ultrasound beams with respect to the heart or at least a portion of the heart. Further, the electronic flex cable inside the catheter shaft may be configured to receive at least one signal from the plurality of pMUT array elements 404 based on transmitting and receiving at least one ultrasound beam of the ultrasound beams. The pMUT imaging and mapping device 302 may be further configured to construct at least one image of at least the portion of the heart based on the at least one signal. It may be noted that the electronic flex cable may be configured to the transmit beamformer 314 and the receive beamformer 312 to display a two-dimensional (2D) image information of the heart or the at least portion of the heart.

In one embodiment, the plurality of pMUT array elements 404 may correspond to MEMS based pMUTs. The catheter shaft may be connected to the handle assembly 324 at one end and to the MEMS based pMUT array 402 at another end. The electronic flex cable inside the catheter shaft may be in communication with the at least one signal trace. It may be noted that the electronic flex cable may be further communicate to the transmit beamformer 314 and the receive beamformer 312, via the dongle 316 to display a two-dimensional (2D) image information of the heart to be scanned.

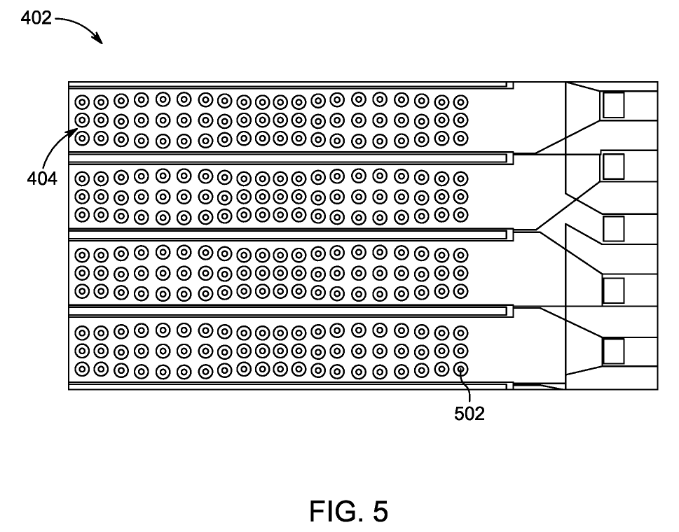

Referring to FIG. 5, a sectional view of a distal end of the pMUT imaging and integrated LAA, ASD, TAVR, TMVR, and TTVR assembly 304 with the plurality of pMUT array elements 404 is disclosed, according to an embodiment of the present disclosure.

The MEMS based pMUT array 402 may comprise the plurality of pMUT array elements 404, arranged towards the distal end of the pMUT imaging and integrated LAA, ASD, TAVR, TMVR, and TTVR assembly 304. The distal end of the pMUT imaging and integrated LAA, ASD, TAVR, TMVR, and TTVR assembly 304 may be provided with the MEMS based pMUT array 402 having the plurality of pMUT array elements 404. Further, each of the plurality of pMUT array elements 404 may have a plurality of individual transducer cells 502 arranged in a manner to provide a wide bandwidth of the individual focused beam. In one embodiment, the MEMS based pMUT array 402 may be constructed from a pMUT array containing individual elements of different diameters. In one embodiment, to achieve wider bandwidth with pMUT arrays, multiple diameters of pMUT cells may be integrated into one element. It may be noted that by arranging pre-shaped pMUTs with different diameters, a broader bandwidth may be realized through the complex interaction between the individual pMUT elements. In one embodiment, the pMUT cells of multiple diameters may achieve a bandwidth of greater than 55%. For example, in 3 elements, there are 5 different dome diameters, and each array is of a different size, such as 300µm.

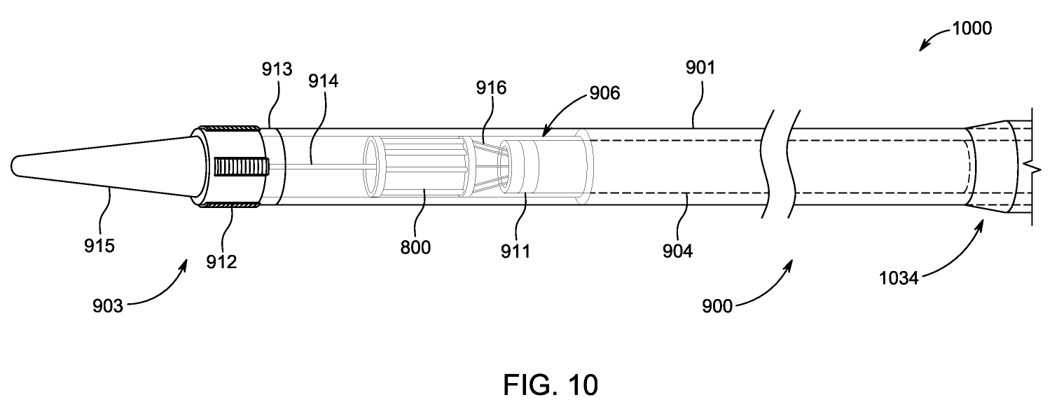

Further, the MEMS based pMUT array 402 may correspond to pMUT and the plurality of pMUT array elements 404 may correspond to a plurality of pMUT elements. In one embodiment, the plurality of pMUT elements may be directed to transmit and receive, the ultrasound beams having the bandwidth including the predetermined fundamental mode vibration of each of the plurality of pMUT elements, such that a single pMUT element may transmit and receive multiple fundamental mode vibrations simultaneously. Further, the electronic flex cable inside the catheter shaft receives the at least one signal from the plurality of pMUT elements. It may be noted that the at least one signal may correspond to the at least one ultrasound beam. The at least one signal may be transmitted to the pMUT imaging and mapping device 302 for further processing in the image processor 310. The image processor 310 may construct the at least one image of the heart. It may be noted that the plurality of pMUT elements may be used to create the individual focused beam. In one embodiment the pMUT elements are arranged in a linear fashion. In a second embodiment the pMUT elements are arranged in cylindrical fashion, as shown in FIGS. 10-11 and 13.

Referring to FIG. 6, a front view of the heart chambers and valves is illustrated. The heart includes four valves as an aortic valve 604, a mitral valve 603, a pulmonary valve 613 and a tricuspid valve 612. These four valves open and close to help move blood from one area to another. The mitral valve 603 and the tricuspid valve 612 move blood from upper chambers of the heart from right atrium 611 and left atrium 602 to lower chambers of the heart that is right ventricle 614 and left ventricle 605, respectively. The aortic valve 604 and the pulmonary valve 613 move blood to the lungs and the rest of the body through the ventricles. It may be noted that when these valves open and close, they create sounds referred as heartbeats.

In general, blood circulation through the heart involves, at first, blood returns from the body to the right atrium 611. It may be noted that the returned blood has been depleted of oxygen when the oxygen was delivered to the body tissues, so it is seeking more oxygen to keep the process going. The right atrium 611, now full of oxygen-depleted blood, pumps the blood through the tricuspid valve 612 into the right ventricle 614. Then the right ventricle 614 contracts to pump blood through the pulmonary valve 613 into a pulmonary artery 601. The pulmonary artery 601 brings blood away from the heart to the lungs where the blood receives the oxygen a user breathes, becoming oxygen-rich blood.

Successively, oxygen-rich blood is returning from the lungs by way of the left atrium 612. The left atrium 602 then moves the blood through the mitral valve 603 into the left ventricle 605. When the left ventricle 605 contracts, it moves blood through the aortic valve 604 into the aorta. The aorta then provides blood to the rest of the body.

Further, the operation of these valves is to move the blood through the heart. For example, when the left atrium 602 and the right atrium 611 chambers contract, the tricuspid valve 612 and the mitral valves 603 open, which both allow blood to move to the left ventricle 605 and the right ventricle 614. Successively, when the two ventricle chambers, the right ventricle 614 and the left ventricle 605, contract, they force the tricuspid valve 612 and the mitral valves 603 to close as the pulmonary valve 613 and the aortic valve 604 open. The blood that is meant to leave the right ventricle 611 and the left ventricle 605, to travel to the body is supposed to be prevented from flowing in the wrong direction by the parts of the aortic valve 604 and the pulmonary valve 613 called the cusps. The cusps help the valves create a tight seal, which helps blood flow in the correct direction. Most heart valve disease occurs in the valves on the left side of the heart, such as, the aortic valve 604 and the mitral valve 603. However, any heart valve may be affected by valve disease.

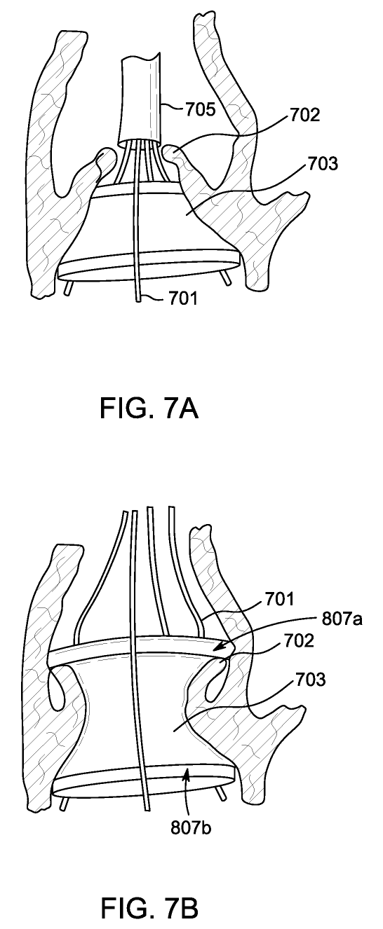

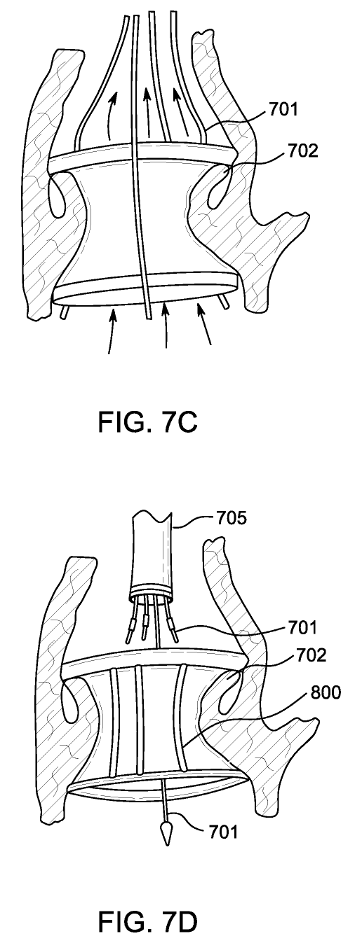

Referring to FIGS. 7A-7D, partially deploying and positioning an artificial valve implant is disclosed, according to an embodiment of the present disclosure.

An inflation channel (not shown) is filled partially, allowing the distal portion of the implant 703 to open to approximately its full diameter. The implant 703 is then pulled back into position at or near the native valve 702 annulus (FIG. 7B). In some embodiments, the distal toroid 807b is at least partially inflated first, and the cardiovascular prosthetic implant 703 is then retracted proximally for positioning the cuff across the native valve 702. The implant 703 may then be fully deployed and released from the control wires 701.

The implant 703 may also be deflated or partially deflated for further adjustment after the initial deployment. As shown in FIG. 7A, the implant 703 may be partially deployed and the PFL tubes 701 used to seat the implant 703 against the native aortic valve 702. The implant 703 may then be fully deployed as shown in FIG. 7B and then tested as shown in FIG. 7C. The implant 703 may then be fully deployed and released from the control wires as shown in FIG. 7D.

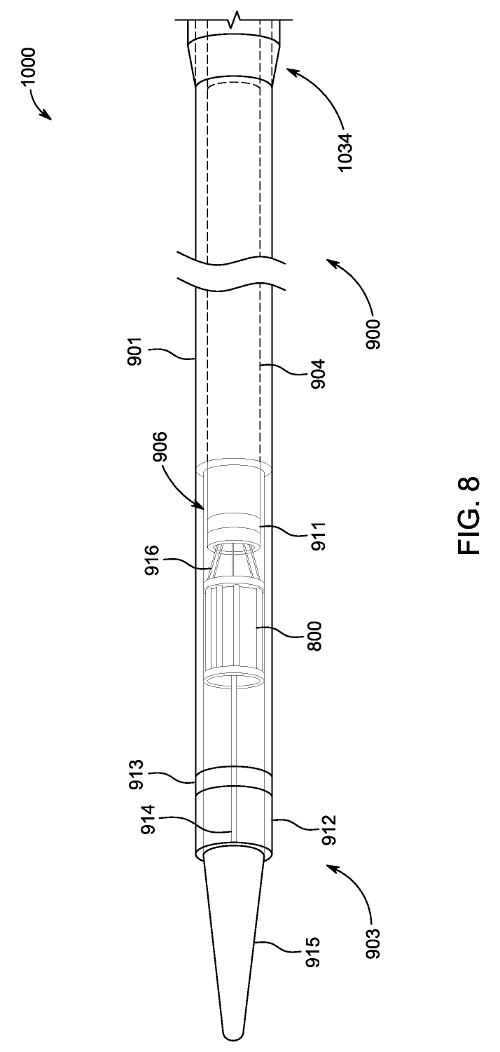

Referring to FIG. 8, a side perspective view of the pMUT imaging and integrated TAVR, TMVR, TTVR assembly 1000 having an introducer catheter 1034 and a deployment catheter 900 with an implant 800 retracted, is disclosed. FIG. 8 is described in conjunction with FIGS. 6-7 and 9-11.

The introducer catheter 1034 may be placed on top of the deployment catheter 900. The pMUT imaging and the TAVR, TMVR, TTVR assembly 1000 includes an outer tubular member 901 that has a proximal end (not shown) and a distal end 903. It also has an inner tubular member 904 with a proximal end (not shown) and a distal end 906 that extends through the outer tubular member 901 in a coaxial manner. The inner tubular member's proximal and distal ends, 906 go beyond the proximal and distal ends 903 of the outer tubular member 901. The outer tubular member's distal end 903 has a position linear array 912, which could be made of Polyvinylidene Fluoride tubing in one example. The implant 800 may be held in a retracted state inside the position linear array 912 until it reaches the implantation site. In addition, there may be an outer sheath marking band 913 located at the distal end 903 of the outer tubular member 901.

Preferably, the position linear array 912 may have a larger outside diameter compared to the adjacent or nearby section of the outer tubular member 901. Another option is for the position linear array 912 to consist of separate tubular components that are attached or connected to each other. Another embodiment involves expanding the outer tubular member 901 to form the larger diameter position linear array 912, resulting in a stem region and the position linear array 912 being formed. from a common tubular member. It should be noted that the stem region's diameter may be reduced.

Further, the proximal end of the inner tubular member 904 may be connected to a handle (not shown) for grasping and moving the inner tubular member 904 with respect to the outer tubular member 901. The proximal end of the outer tubular member 901 may be connected to an outer sheath handle (not shown) for grasping and holding the outer tubular member 708 stationary with respect to the inner tubular member 904.

Further, the inner tubular member 904 comprises a multi-lumen hypo-tube. In one embodiment, a neck section (not shown) may be located at the proximal end of the inner tubular member 904. The neck section may be made from a material selected from a group of materials of stainless steel, Nitinol or another suitable material to serve additional strength for moving the inner tubular member 904 within the outer tubular member 901. Further, the pMUT imaging and TAVR, TMVR, TTVR assembly 1000 may comprise a marker band 911 is present at the distal end 906 of the inner tubular member 904.

Hereinafter, the deployment catheter 900 may be referred to as a delivery catheter. The delivery catheter 900 may be advanced to a position proximate a native valve (not shown). In one embodiment, the pMUT imaging and the TAVR, TMVR, TTVR assembly 1000, including both the introducer catheter 1034 and the delivery catheter 900 may be advanced to a position proximate the native valve. Further, after the delivery catheter 900 is advanced over an aortic arch and past the aortic valve 604, the position of the outer tubular member 901 relative to the introducer catheter 1034 may be maintained by adjusting seal assembly.

Further, the inner tubular member 904 may comprise at least four lumens, with one accommodating a guidewire tubing 914. Further, each of the other lumens accommodate a positioning-and-fill lumen (PFL) tubing 916. The guidewire tubing 914 may be configured to receive a guidewire. The PFL tubing 916 may be configured to function both as a control wire for positioning the implant 800 at the implantation cite, and as an inflation tube for delivering a liquid, gas or inflation media to the implant 800. It may be noted that the PFL tubing 916 allows an angular adjustment of the implant 800.

The guidewire tubing 914 may be longer than and may extend throughout the length of the delivery catheter 900. The guidewire tubing 914 may have a proximal end passing through an inner sheath handle (not shown) for operator's control, and a distal end extending past the distal end 903 of the outer tubular member 901. The distal end of the guidewire tubing 914 may be coupled to a guidewire tip 915. The guidewire tip 915 may close the distal end 903 of the outer tubular member 901 and protect the retracted implant 800, for example, during the advancement of the delivery catheter 900. Further, the guidewire tip 915 may be distanced from the outer tubular member 901 by proximally retracting the outer tubular member 901 while keeping the guidewire tubing 914 idle. Alternatively, the guidewire tubing 914 may be advanced while holding the outer tubular member 901 stationary.

Referring to FIG. 8, the handle of the deployment catheter 900 and the implant 800. By moving all of the PFL tubing 916 together or the inner tubular member 904, the implant 800 may be advanced or retracted in a proximal or distal direction. By advancing only a portion of the PFL tubing 916 relatives to the other PFL tubing 916, the angle or orientation of the implant 800 may be adjusted relative to the native anatomy. Radiopaque markers on the implant 800 or on the PFL tubing 916, or the radio-opacity of the PFL tubing 916 themselves, may help to indicate the orientation of the implant 800 as the operator positions and orients the implant 800.

In some embodiments, the implant 800 is fully inflated by pressurizing the endoflator attached to a first PFL tube of the PFL tubing 916 that is in communication with the first inflation valve (not shown) that leads to the proximal toroid 807a, while the endoflator attached to the second inflation valve (not shown) that is in communication with the distal toroid 807b is closed. The fluid or gas may flow into the distal toroid 807b through the one-way check valve. The proximal toroid 807a is then deflated by de-pressurizing the endoflator attached to the second inflation valve. The distal toroid 807 b will remain inflated because the fluid or gas cannot escape through the check valve (not shown). The implant 800 may then be positioned across the native annulus. Once in the satisfactory placement, the proximal toroid 807a may then be inflated again.

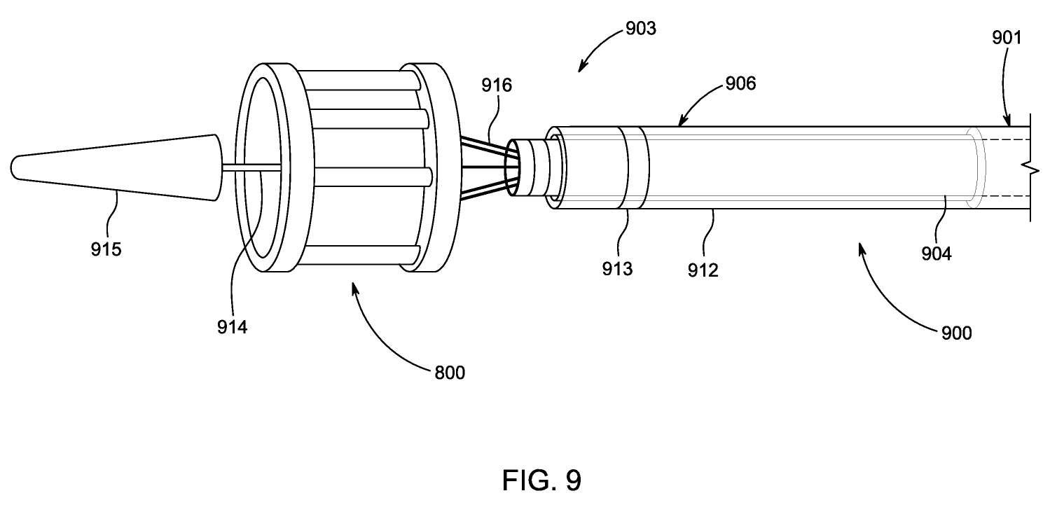

Referring to FIG. 9, the implant 800 may be revealed or exposed by retracting the outer tubular member 901 partially or completely while holding the inner tubular member 904 stationary and allowing proper placement at or beneath the native valve. In some embodiments, the implant may also be revealed by pushing the inner tubular member 904 distally while holding the outer tubular member 901 stationary. Once the implant 800 is unsheathed, it may be moved proximally or distally, and the fluid or inflation media may be introduced to a cuff (not shown) providing shape and structural integrity. In some embodiments, the distal toroid of the inflatable cuff or inflatable structure is inflated first with a first liquid, and the implant 800 is positioned at the implantation cite using the links between the implant 800 and the combined delivery system. In some embodiments, no more than three links are present. In some embodiments, the links are PRL tubes 916, which may be used to both control the implant 800 and to fill the inflatable cuff. The implant 800 may be otherwise inflated or controlled using any of the other methods disclosed above.

Referring to FIG. 10, a view of the pMUT imaging and the TAVR, TMVR, TTVR assembly 1000 with the with a plurality of linear pMUT imaging arrays (not shown) arranged in linear fashion, is disclosed, according to an embodiment of the present disclosure. The plurality of linear pMUT imaging arrays, are mounted over the position linear array 912 towards the distal end 903 of the outer tabular member 901. The plurality of linear pMUT imaging arrays are configured to direct each of the plurality of MEMS based pMUT array elements 404, via the at least one signal trace, to transmit and receive, with respect to heart, ultrasound beams. Further, receive at least one signal from the plurality of MEMS based pMUT array elements 404 based on transmitting and receiving at least one ultrasound beam of the ultrasound beams. Successively, construct at least one image of at least a portion of the heart based on at least one signal.

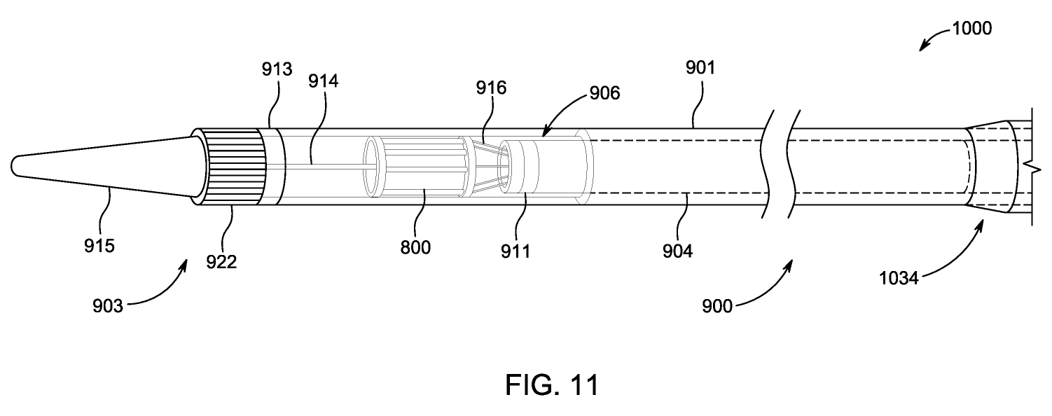

Referring to FIG. 11, a view of the pMUT imaging and the TAVR, TMVR, TTVR assembly 1000 with one or more circular pMUT imaging arrays (not shown) arranged in linear fashion, is disclosed, according to an embodiment of the present disclosure.

The one or more circular pMUT imaging arrays, are mounted over the position circular array 922 towards the distal end 903 of the outer tabular member 901. The one or more circular pMUT imaging arrays are configured to direct each of the plurality of MEMS based pMUT array elements 404, via the at least one signal trace, to transmit and receive, with respect to heart, ultrasound beams. Further, receive at least one signal from the plurality of MEMS based pMUT array elements 404 based on transmitting and receiving at least one ultrasound beam of the ultrasound beams. Successively, construct at least one image of at least a portion of the heart based on at least one signal.

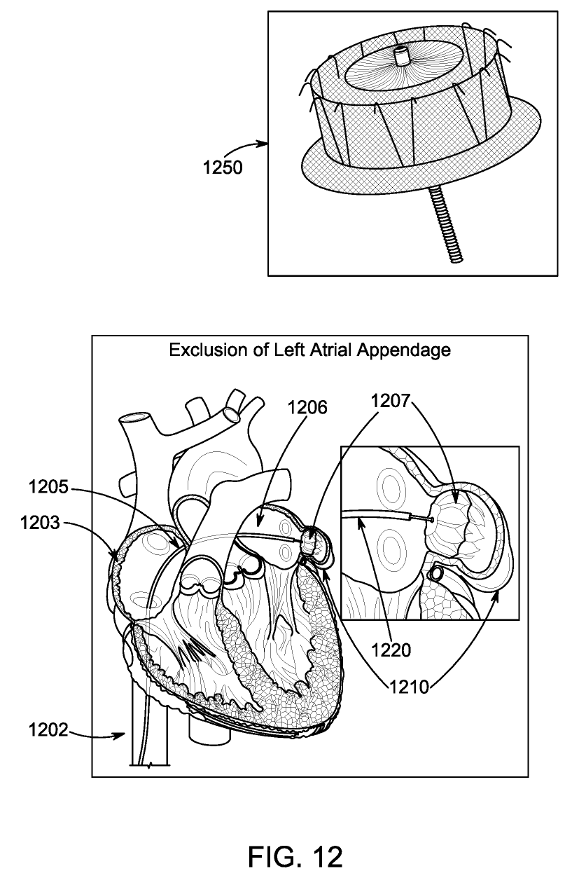

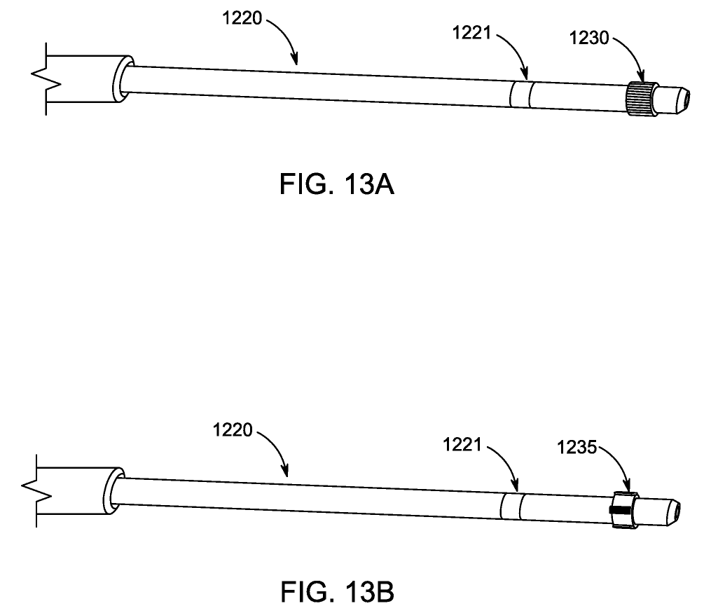

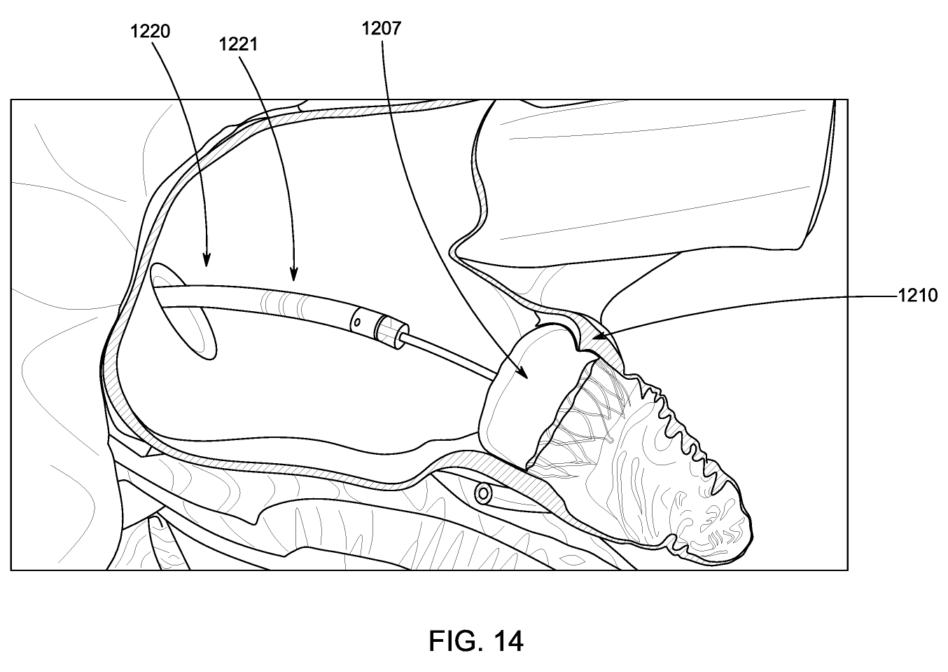

Referring to FIG. 12, heart chambers and valves, with a Watchman 1207 or Amulet 1250 deployed into an LAA 1210 are illustrated. FIGS. 13A-13B and 14 are described in conjunction with FIG. 12.

The heart includes an inferior vena cava 1202, a right atrium 1203, an interatrial septum 1205, a left atrium 1206 and the LAA 1210. The inferior vena cava 1202 is a large vein that carries the deoxygenated blood from the lower and middle body into the right atrium 1203 of the heart. The interatrial septum 1205 is a septum that lies between the left atrium 1206 and the right atrium 1203 of the heart. The LAA 1210 is a small pouch extending off the side of the left atrium 1206 in the heart.

A guide catheter (not shown) is placed through the inferior vena cava 1202 into the right atrium 1203 of the heart. The guide catheter is further placed across the interatrial septum 1205 into the left atrium 1206. Further, the guide catheter is positioned near the LAA 1210. The LAA 1210 may also be referred to as the LAA ostium 1210. The guide catheter is then advanced over to guide into the LAA 1210. A marker band 1221, as shown in FIGS. 13A-13B, is used to guide the guide catheter placement under fluoroscopy. The marker band 1221 may be a radiopaque marker. Further, additional guidance to the guide catheter is provided by a pMUT circular array 1230, as shown in FIG. 13A. The pMUT circular array 1230 may be replaced by a pMUT linear array 1235, as shown in FIG. 13B, to provide additional guidance to the guide catheter. In some embodiments, the transcatheter valve replacement assembly comprises a catheter having 1 to a plurality of pMUT linear arrays. In some embodiments, the pMUT linear array 1235 may be 1 to 4 arrays and the pMUT circular array 1230 may be one array.

Further, the Watchman 1207 is then pushed through a catheter sheath 1220. An amulet 1250 may also be used in place of the Watchman 1207. The Watchman 1207 is further deployed into the LAA 1210, as shown in FIG. 14. Once the placement of the guide catheter is satisfactory, the Watchman 1207 or the Amulet 1250 by expansion or other means after which the catheter sheath 1220 is removed.

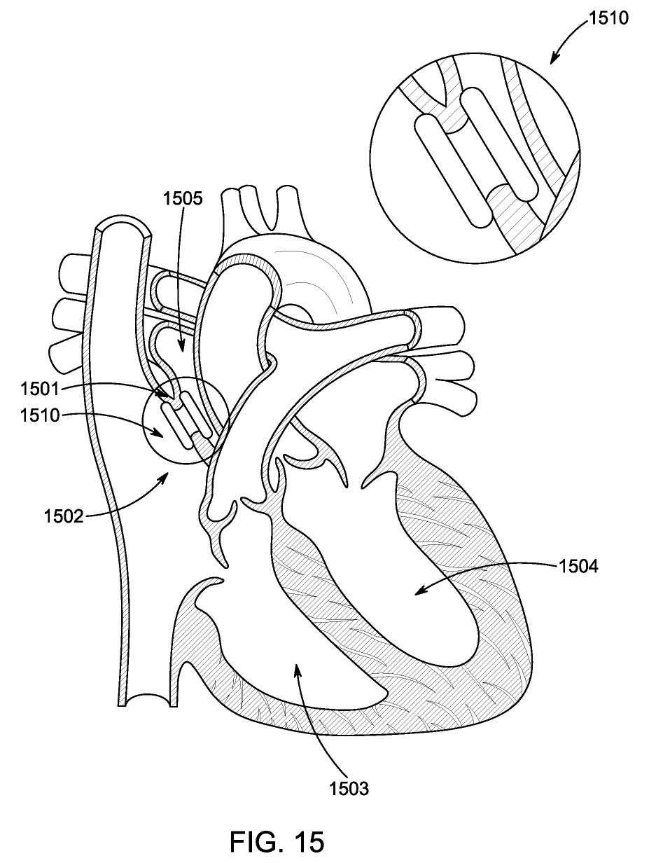

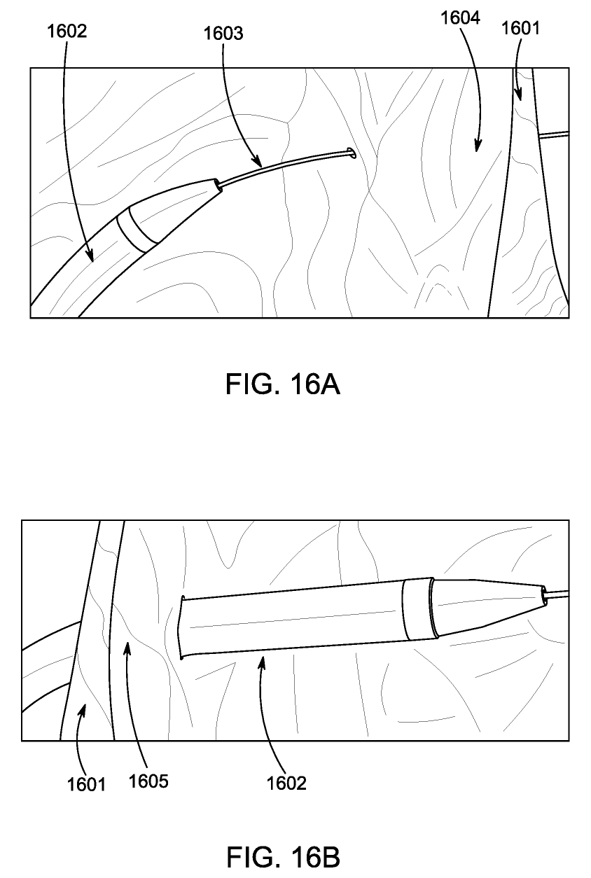

Referring to FIG. 15, heart chambers with an Arterial septum closure device 1510 is illustrated. FIGS. 16A-16B is described in conjunction with FIG. 15.

The heart further includes an Atrial Septum wall 1501, a right atrium 1502, a right ventricle 1503, a left ventricle 1504 and a left atrium 1505. The right atrium 1502 and the right ventricle 1503 are often referred to together as the right heart. The left atrium 1505 and the left ventricle 1504 are together referred to as the left heart. In some embodiments, the ASD is a birth defect of the heart in which there is a hole in the Atrial Septum wall 1501. Further, a view of the hole in the Atrial Septum wall 1501 is illustrated in FIG. 15. The Atrial Septum wall 1501 is the wall between the right atrium 1502 and the left atrium 1505. Closing the hole in the Atrial Septum wall 1501 opening keeps blood in the left atrium 1505 separate from the blood in the right atrium 1502. Closing the hole prevents blood flow containing oxygen from the left atrium 1505 into the right atrium 1502. The hole is closed with the Arterial septum closure device 1510 to prevent blood flow containing oxygen from the left atrium 1505 into the right atrium 1502.

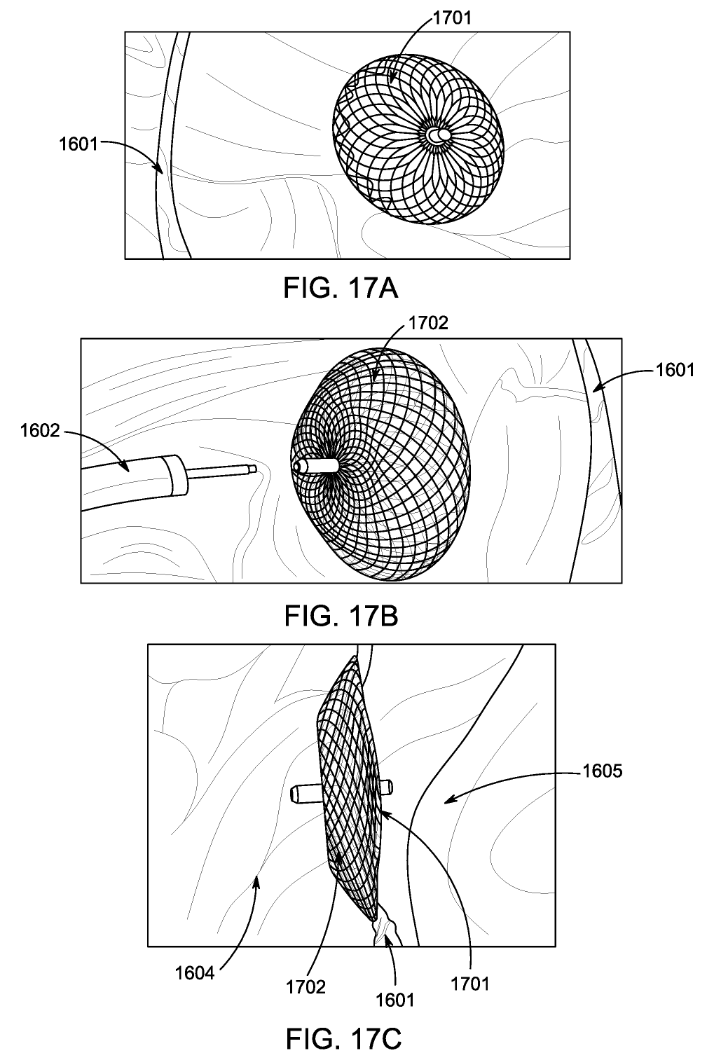

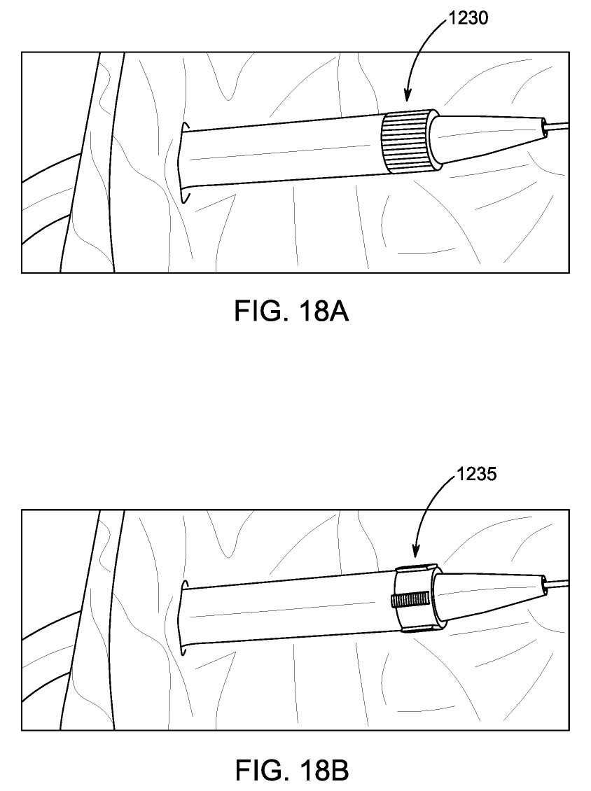

Referring to FIGS. 16A-16B, deployment of the Arterial septum closure device 1510 is illustrated. FIGS. 17A-17C and 18A-18B are described in conjunction with FIGS. 16A-16B.

The Arterial septum closure device 1510 used may include at least an amulet. Further, the Arterial septum closure device 1510 may be used interchangeably with the amulet 1510.

Referring to FIGS. 16A-16B, a catheter 1602 is illustrated. The catheter 1602 further includes both sides of the amulet 1510. The both sides of the amulet 1510 further includes a distal side of the amulet 1510 and proximal side of the amulet 1510. The both sides of the amulet 1510 is further moved along the guide wire 1603 from the right atrium 1604 through the Atrial Septal wall 1601 into the left atrium 1605.

FIG. 17A illustrates the distal side of the amulet 1510 deployed and brought back against the left atrium 1605 side of the Atrial Septal Wall 1601. The distal side may include the left atrium half 1701 of the amulet 1510. FIG. 17B illustrates the proximal side of the amulet 1510 deployed against the right atrium 1604 side of the Atrial Septal Wall 1601. The proximal side may include the right atrium half 1702 of the amulet 1510. The catheter 1602 is detached from the left atrium half 1701 of the amulet 1510 and removed from the heart.

FIG. 17B further illustrates a successful deployment of the amulet 1510. The Atrial Septal Wall 1601 is sealed by the compression of the left atrium half 1701 of the amulet 1510 and the right atrium half 1702 of the amulet 1510. Further, a side perspective view of the deployed Arterial septum closure device 1510 or the Amulet 1510 is illustrated in FIG. 17C. FIG. 17C shows the deployed amulet 1510 to the Atrial Septal Wall 1601 between the right atrium 1604 and the left atrium 1605.

Referring to FIGS. 18A-18B, the addition of PMUT imaging to the catheter 1602 is illustrated. The catheter 1602 with the pMUT circular array 1230 is illustrated in FIG. 18A. The catheter 1602 with the pMUT linear array 1235 is illustrated in FIG. 18B.

In some embodiments, an integrated TTVR may be disclosed. The integrated TTVR may involve patching holes or tears in the valve flaps. The valve flaps may reconnect leaflets or cusps. Further, reshaping or removing excess valve tissue from the reconnected valve flaps so that the leaflets or cusps may close tightly. Further, replacing cords that support the valve to repair the structural support. Further, separating valve flaps that have fused. Successively, tightening or reinforcing the ring around the valve (annulus).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth here below not be construed as being order-specific unless Such order specificity is expressly stated in the claim.

What is claimed is:

1. An integrated transcatheter valve replacement and Piezoelectric Micro-Machined Ultrasonic Transducer (pMUT) ultrasonic imaging system, the system comprising:
a transcatheter valve replacement assembly having a longitudinal axis, a proximal end, and a distal end, wherein the transcatheter valve replacement assembly comprises a self-expanding valve, a frame and leaflets; and
a micro-electromechanical (MEMS) based pMUT transducer having MEMS based pMUT array disposed within the distal end of the transcatheter valve replacement assembly, wherein the MEMS based pMUT array comprises a substrate and a plurality of pMUT array elements arranged on the substrate.

2. The system of claim 1, wherein the transcatheter valve replacement is a transcatheter aortic valve replacement (TAVR) catheters, transcatheter mitral valve replacement (TMVR) catheters, or transcatheter tricuspid valve replacement or repair (TTVR) catheter.

3. The system of claim 1, further comprising a catheter shaft connected at one end to a handle assembly and at other end to the MEMS based pMUT array and the transcatheter valve replacement assembly.

4. The system of claim 3, wherein the MEMS based pMUT array and the transcatheter valve replacement assembly comprises a steering control unit positioned within the handle assembly, for articulating a distal tip of the transcatheter valve replacement assembly and aligning face of the MEMS based pMUT array towards internal views including an anterior position or a posterior position and right or left position of the tissue.

5. The system of claim 4, wherein the distal tip of the MEMS based pMUT array is coated with a material to provide electrical isolation and transmission of ultrasound signals.

6. The system of claim 1, wherein the MEMS based pMUT array and the transcatheter replacement assembly is coupled to a dongle, and the dongle is configured to communicate ultrasound transmit pulses and ultrasound receive waveforms between the pMUT array and the ultrasound imaging system.

7. The system of claim 1, wherein each of the plurality of pMUT array elements having transducer cells of multiple diameters, to achieve a wide bandwidth.

8. The system of claim 1, wherein each of the plurality of pMUT array elements is a linear phased array.

9. The system of claim 1, wherein the transcatheter valve replacement assembly comprises a catheter having 1 to a plurality of pMUT linear arrays.

10. The system of claim 9, wherein the catheter comprises a pMUT circular array.

11. The system of claim 1, wherein the system is deployed into the heart chambers.

12. An integrated transcatheter valve replacement and Piezoelectric Micro-Machined Ultrasonic Transducer (pMUT)ultrasonic imaging system, the system comprising:
a transcatheter valve replacement assembly having a longitudinal axis, a proximal end, and a distal end;
a micro-electromechanical (MEMS) based pMUT array disposed within the distal end of the transcatheter valve replacement assembly, wherein the MEMS based pMUT array comprises a substrate and a plurality of MEMS based pMUT array elements arranged on the substrate; and
an electronic flex cable connected at one end to a handle assembly and at other end to the MEMS based pMUT array, wherein the electronic flex cable is in communication with at least one signal trace, and is configured to:
direct each of the plurality of MEMS based pMUT array elements, via the at least one signal trace, to transmit and receive, with respect to heart, ultrasound beams;
receive at least one signal from the plurality of MEMS based pMUT array elements based on transmitting and receiving at least one ultrasound beam of the ultrasound beams; and
construct at least one image of at least a portion of the heart based on the at least one signal.

13. The system of claim 12, where the transcatheter valve replacement is a transcatheter aortic valve replacement (TAVR) catheters, transcatheter mitral valve replacement (TMVR) catheters, or transcatheter tricuspid valve replacement or repair (TTVR) catheter.

14. The system of claim 12, wherein the ultrasound beams having a bandwidth including a predetermined fundamental mode vibration of each of the plurality of pMUT array elements, such that a single array element transmits and receives multiple fundamental mode vibrations simultaneously.

15. A method of positioning a prosthetic implant within a heart, the method comprising:
advancing together a delivery catheter and an introducer catheter preassembled over the delivery catheter into a patient's vascular system;
advancing the prosthetic implant to a position proximate a native valve of the heart, the prosthetic implant being at least partially disposed within a distal end of the delivery catheter during advancement of the introducer catheter; and
deploying the prosthetic implant.

16. The method of claim 15, wherein the delivery catheter comprises the prosthetic implant and a distal tip inserted directly into the access vessel to dilate the distal tip to the access vessel for the introducer catheter.

17. The method of claim 15, wherein during advancement, an outer diameter of the distal end of the delivery catheter is greater than an inner diameter of a distal end of the introducer catheter.

18. The method of claim 17, wherein the introducer catheter comprises a hemostasis valve assembly at a proximal end of the introducer catheter.

19. The method of claim 15, wherein the delivery catheter is advanced over an aortic arch and past the aortic valve, the position of an outer tubular member of the delivery catheter relative to the introducer catheter is maintained by adjusting a seal assembly.

20. An integrated transcatheter tricuspid valve replacement or repair (TTVR) and Piezoelectric Micro-Machined Ultrasonic Transducer (pMUT) ultrasonic imaging system, the system comprising:
a transcatheter TTVR assembly having a longitudinal axis, a proximal end, and a distal end, wherein the transcatheter TTVR assembly comprises a self-expanding valve, a frame and leaflets; and
a micro-electromechanical (MEMS) based pMUT transducer having MEMS based pMUT array disposed within the distal end of the transcatheter TTVR assembly, wherein the MEMS based pMUT array comprises a substrate and a plurality of pMUT array elements arranged on the substrate.
